(19) United States
(12) Patent Application Publication
(10) Pub. No.: US 2022/0031535 A1
(43) Pub. Date: Feb. 3, 2022

(54) A FIRST-AID KIT CONTAINER DISPENSING SYSTEM

(71) Applicant: SCATTERBRAIN PTY LTD, West End (AU)

(72) Inventor: Christopher Thomas CLARKE, West End (AU)

(21) Appl. No.: 17/309,433

(22) PCT Filed: Nov. 27, 2019
(86) PCT No.: PCT/AU2019/051298

§ 371 Date: May 26, 2021

(30) Foreign Application Priority Data
Nov. 27, 2018 (AU) 2018904504

(51) Int. Cl. A61F 17/00; A47F 7/28; A47F 1/04; G16H 40/40; G06Q 10/08; G06K 7/10; G06K 19/07

(52) U.S. Cl. CPC A61F 17/00; A47F 7/28; A47F 1/04; A47B 67/02; G06Q 10/087; G06K 7/10366; G06K 19/0723; G16H 40/40

(57) ABSTRACT

A first-aid kit dispensing system has a plurality of cradles for a respective plurality of first-aid kit containers. Each container may have a container body and container lid a plurality of first-aid kit items therein. At least one sensor is configured for detecting the presence of the containers and a controller operably coupled to the at least one sensor. The controller may have a memory device recording an inventory of a plurality of first aid items. In use, the controller may be configured for using the at least one sensor to detect at least one of a container identity and a container type of a container inserted into or removed from a container and accessing the inventory data to identify at least one first aid item of the container.

### FIELD OF THE INVENTION

This invention relates generally to a first-aid kit dispensing system and, more particularly, first-aid kit dispensing system comprising a plurality of cradles each having a first-aid kit container removably retained therein.

### BACKGROUND OF THE INVENTION

U.S. Pat. No. 8,341,041 B2 (Hull) 13 Nov. 2011 discloses a method and apparatus for automated medical supply take/store tracking which comprises inner and outer wireless sensors. Inventory items stored within a cabinet are tagged with a radiofrequency tags and depending on the sequence of sensing thereof by the sensors, the system is able to identify whether an item is taken or stored.

U.S. Pat. No. 6,539,281B2 (Wan) 25 Mar. 2005 discloses a similar arrangement wherein radiofrequency tags are used to identify items stored within a medical supply cabinet for retrieving information regarding medications across the Internet.

The present invention seeks to provide a system, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a first-aid kit dispensing system comprising a plurality of cradles for a respective plurality of first-aid kit containers.

Each container may have a container body and container lid and a plurality of first-aid kit items therein.

At least one sensor is configured for detecting the presence of the containers and a controller is operably coupled to the at least one sensor. The controller comprises a memory device recording an inventory of a plurality of first aid items. The controller may use the at least one sensor to detect at least one of a container identity and a container type of a container inserted into or removed from a container to access the inventory data to identify at least one first aid item of the container.

The present system may comprise a wall mounted rack having the plurality of cradles vertically mounted therein and having different types of first-aid kits containers for various first-aid situations, such as wounds, burns, sprains, serious wounds, minor wounds and the like.

The present system may for example be installed in an office environment for ready access when required for various first-aid scenarios.

The system may monitor the opening of container lids to determine when replenishment of first-aid kit items as required. In embodiments, the system comprises an electrical circuit interfacing the container body and lid, such as a tear-away foil strip which is broken by the removal of the lid. Each cradle may comprise electrical contacts that make contact with the electrical circuit to detect the removal of the lid.

In embodiments, the system may detect the removal or access of individual first-aid items within each container. In this regard, each container may comprise a plurality of electrical circuits interfacing each first-aid item therein which is configured to break when each first-aid item is accessed or removed. For example, the container may comprise a plurality of compartment lids which break each respective electric circuit when opened. Each cradle may comprise electrical contacts for each electric circuit such that the controller is able to identify which items have been accessed and therefore require replenishment.

Alternatively, each container may comprise a plurality of radiofrequency tags, antennas thereof connected in series with a plurality of electric circuits and wherein each electric circuit breaks when a respective first-aid item is accessed thereby disabling a respective radiofrequency tag. As such the controller is configured for using a radio frequency receiver to detect which of the plurality of radio frequency tags are disabled to identify which first-aid items have been accessed.

In embodiments, the containers and cradles are keyed such that each type of container can only be inserted into a corresponding keyed cradle. The containers and cradles may comprise unique keying formations which interlock when the container is inserted into a corresponding cradle. The at least one sensor may comprise a plurality of sensors for the keying formations which detects interlocking thereof.

The system may use a sensor for each cradle (such as a light beam or electromechanical switch sensor) or alternatively utilise a radiofrequency reader to detect proximity and, in embodiments, read data from radiofrequency tags of each container. Each radiofrequency tag may encode the container and/or container type which is interrogated by the controller.

The system may comprise a user interface and, in a first-aid situation, the user may use the user interface to input the type of first-aid situation.

As such, the controller may use the user input information and the inventory data to identify at least one container for the first-aid situation. The controller may also output instructional information via the user interface.

In embodiments, the user interface may use a drill down interface to provide first-aid situation specifics, such as for example, specifying a burn first-aid situation and further specifying a minor burn on the right-hand arm.

According to one aspect, there is provided a first-aid kit dispensing system comprising a plurality of cradles for a respective plurality of first-aid kit containers, each container having a container body and container lid a plurality of first-aid kit items therein, at least one sensor configured for detecting the presence of the containers, a controller operably coupled to the at least one sensor, the controller comprising a memory device recording an inventory of a plurality of first aid items and wherein, in use, the controller is configured for using the at least one sensor to detect at least one of a container identity and a container type of a container inserted into or removed from a container and accessing the inventory data to identify at least one first aid item of the container.

Each container may comprise a container lid and a container body and wherein the system may be configured for detecting the removal of the container lid from the container body.

The controller may be configured for updating the inventory data to indicate that the at least one first-aid item requires replenishment when detecting the removal of the container lid.

Each cradle may comprise an indicator and wherein the controller may be configured for controlling the indicator to indicate that the at least one first-aid item requires replenishment when detecting the removal of the container lid.

Each container may comprise an electrical circuit interfacing the container lid and the container body and wherein opening of the container lid breaks the electrical circuit and wherein the system may be configured for detecting breaking of the electrical circuit.

The electrical circuit may comprise tearable foil strips interfacing the container lid and the container body which are torn when the lid may be removed from the body.

The cradle may comprise electrical contacts which make electrical contact with the electrical circuit when a respective container may be placed therein.

The container may comprise an electric circuit for each first-aid item therein and wherein each electric circuit breaks when a respective first-aid item may be accessed and wherein the controller may be configured for determining which first-aid items have been accessed accordingly.

The cradle may comprise electrical contacts for each electric circuit.

Corresponding containers and cradles are keyed such that each container can only be inserted into a correspondingly keyed cradle and wherein the controller may be configured for identifying the at least one of the container identity and container type depending on which container as inserted into or removed from a particular container.

Each keyed container and correspondingly keyed cradle may comprise male and female keying formations on respective abutting surfaces thereof which interlock when the keyed container may be inserted into the correspondingly keyed cradle.

The at least one sensor may comprise a plurality of sensors for each cradle and wherein each of the plurality of sensors may be configured for detecting interlock of the keying formations.

Each sensor may comprise a light beam interruption sensor operative across the respective key information.

Each cradle may comprise a base and edges sized and shaped according to a size and shape of the container.

The at least one sensor may comprise a plurality of sensors for each cradle and wherein each sensor may comprise a light beam interrupt sensor configured for detecting interruption of a beam of light by a container inserted within a respective cradle.

The at least one sensor may comprise a plurality of sensors for each cradle and wherein each sensor may comprise an electromechanical switch comprising a switch lever which extends into the cradle and which may be deflected by the container when located within the cradle for being indicative of the presence of the container.

Each container may comprise computer readable media encoding the at least one of the container identity and container type and wherein the controller may be configured for interrogating the computer readable media using a radiofrequency receiver to read the at least one of the container identity and container type therefrom.

Each container may comprise a radiofrequency tag and the sensor may comprise a radiofrequency receiver configured for reading the radiofrequency tags.

The container may comprise a plurality of radiofrequency tags, antennas thereof connected in series with a plurality of electric circuits and wherein each electric circuit breaks when a respective first-aid item may be accessed thereby disabling a respective radiofrequency tag and wherein the controller may be configured for using a radio frequency receiver to detect which of the plurality of radio frequency tags are disabled to identify which first-aid items have been accessed.

The controller may be configured for monitoring an expiry date of each container.

The controller may be configured for calculating the expiry date in accordance with the container identity or container type.

The controller may be configured for reading an expiry date encoded within computer readable medium of the container.

The controller may be configured for using the at least one sensor for recording and insertion date when the container may be inserted within a respective cradle and a calculating the expiry date accordingly.

Each cradle may comprise an indicator and wherein the controller may be configured for controlling the indicator according to the expiry date of each respective container.

The indicator panel may comprise an LED adjacent each cradle and wherein each LED may be controllable in at least two states indicative of at least two shelf life states of each container.

The system may comprise a user interface and wherein, in use, the controller may be configured for receiving data via the user interface and identifying at least one container accordingly.

The user interface may comprise a drill down interface allowing for the further input of specifics of a first-aid situation and wherein the controller may be configured identifying at least one container according to the specifics.

The system further may comprise a user interface and wherein, in use, the controller may be configured for detecting a removal of a container and outputting instructional information for the use of the at least one container.

The system further may comprise a notification controller and, wherein, in use, the controller may be configured for identifying at transmitting an electronic notification according to the at least one of the container identity and the container type of the container.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a functional schematic of a first-aid kit dispensing system in accordance with an embodiment;

FIG. 2 shows a cradle for a first-aid kit container in accordance with an embodiment;

FIG. 3 shows a side elevation view of a light beam interrupt sensor configured for detecting the presence or absence of a container in accordance with an embodiment;

FIG. 4 shows a side elevation view of an electromechanical sensor configured for detecting the presence or absence of a container in accordance with an embodiment;

FIG. 5 illustrates an embodiment wherein the system comprises a radiofrequency receiver configured for sensing the presence of radiofrequency tags of each container within a certain proximity in accordance with an embodiment;

FIG. 6 shows container comprising a radiofrequency tag in accordance with an embodiment;

FIG. 7 illustrates the keying of corresponding cradles and containers in an embodiment;

FIG. 8 illustrates a light beam interrupt configured for detecting the interlock of correspondingly keyed cradles and containers in accordance an embodiment;

FIG. 9 illustrates differing keying formations indicative of the purpose of each container in accordance with an embodiment;

FIG. 10 illustrates a top perspective view of the container comprising a breakable electrical circuit indicative of the removal of the lid thereof;

FIG. 11 illustrates an underside view of the container of FIG. 10;

FIG. 12 illustrates a cradle comprise electrical contacts for the container of FIG. 11;

FIG. 13 illustrates a container comprising a plurality of apartments each having a respective circuit indicative of the opening thereof; and

FIG. 14 illustrates a plurality of radiofrequency tags of the container is comprising an antenna connected to a breakable circuit in accordance with an embodiment.

### DESCRIPTION OF EMBODIMENTS

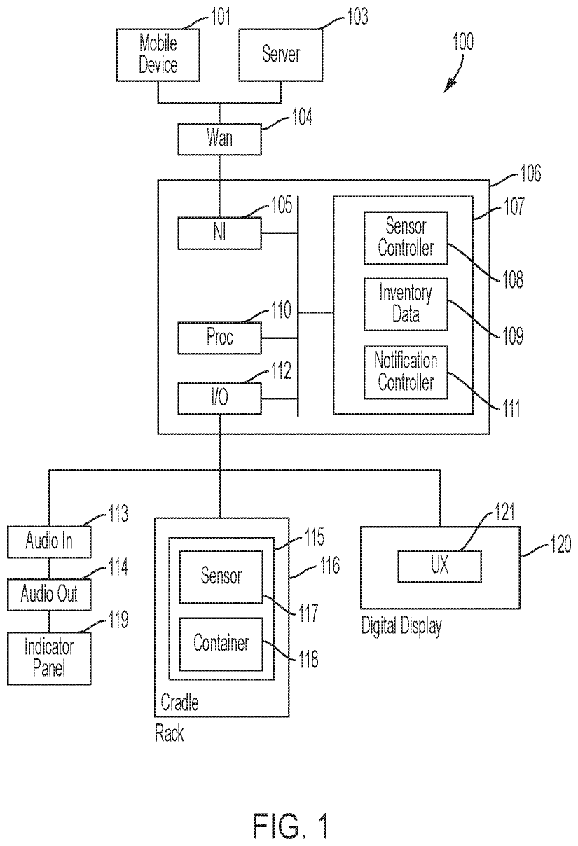

FIG. 1 shows a functional schematic of a first-aid kit dispensing system 100.

The system may comprise a rack 116 comprising a plurality of cradles 115. Each cradle 115 holds a first-aid kit container 118.

The system 100 further comprises at least one sensor 117 configured to detect the presence or absence of the containers 118 within the cradles 115.

In embodiments, each cradle 115 comprises a corresponding sensor 117 as is illustrated in FIGS. 3 and 4. However, in alternative embodiments, the rack 116 comprises a radiofrequency sensor 135 configured for detecting the presence of associated radiofrequency tags 136 of each container 118 is illustrated in FIG. 5. The radiofrequency sensor 135 may be a Radio-frequency identification (RFID) sensor. The radio frequency tags 136 may encode data including container identity, container type, container contents, container contents expiry date(s) and the like.

The controller 106 may comprises a microprocessor 110 for processing digital data. The controller 106 may comprise an I/O interface 112 operably connected to the plurality of sensors 117.

As such, in use, when a particular container 118 is removed from the cradle 115, the controller 106 is configured for identifying which particular container 118 or which type of container 118 has been removed.

The controller 106 may comprise a memory device 107 in operable communication with the microprocessor 110 across a system bus. The memory device 107 may comprise a plurality of computer program code instructions and associated data. In use, the microprocessor 110 may fetch these computer program code instructions and associated data from the memory device 107 for interpretation and execution. The computer program code instructions may be logically grouped into various computer program code controllers including those shown in FIG. 1 in which will be described in further detail below.

In embodiments, the controller 106 may interface with a wide area network 104, such as the Internet, across a data interface and may comprise a network interface 105 therefor. In this way, the controller 106 may interface with a plurality of mobile devices 101 or servers 103, including in the manner described hereunder.

In embodiments, the controller 106 may control a digital display 120 to display digital data thereon and, in embodiments, receive user haptic interface gestures therefrom by way of a touch sensitive user interface overlay.

The digital display 120 may be controlled by the controller 106 to display a user interface 121 including in the manner described hereunder.

The I/O interface 112 may further interface with an audio input device 113, an audio output device 114 and/or and indicator panel 119 in embodiments.

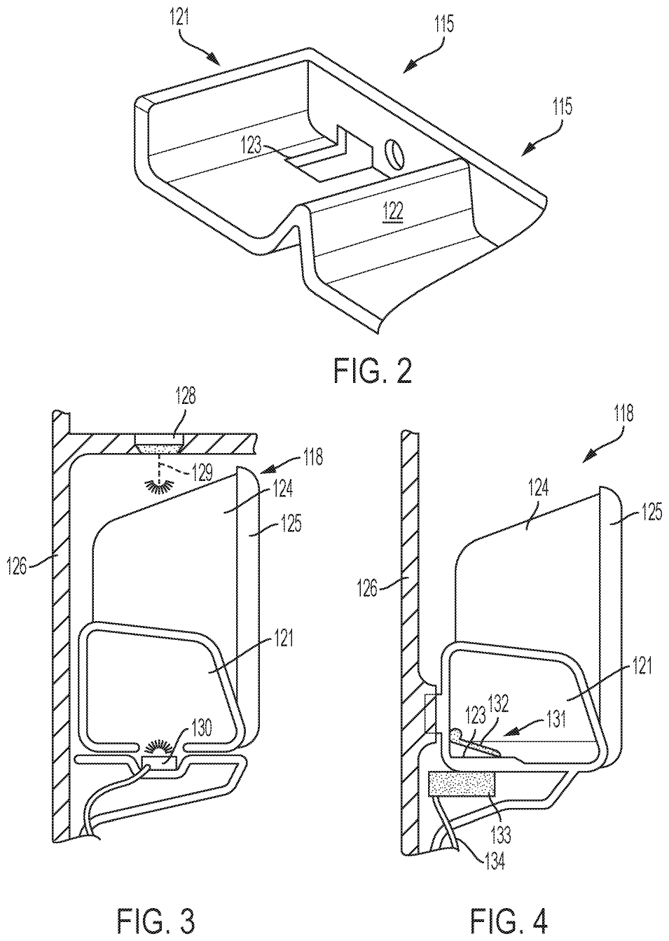

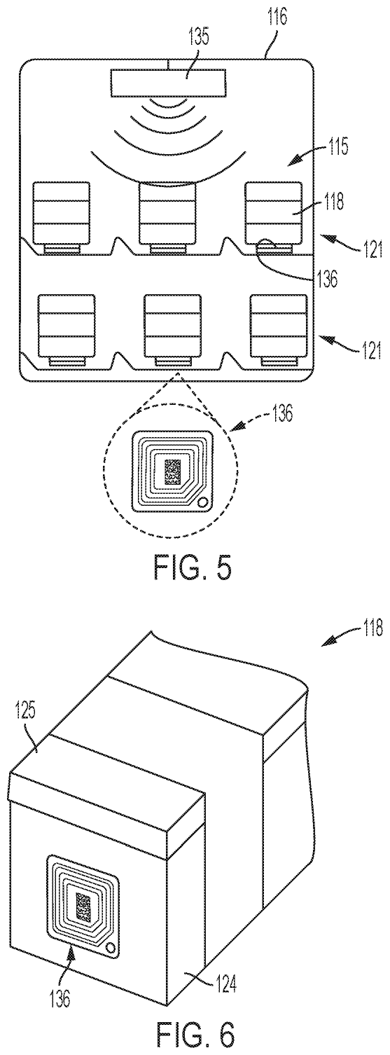

FIG. 2 shows an embodiment wherein the rack 116 comprises a shelf 121 comprising partitions 122 therealong defining a plurality of cradles 115. Each cradle 115 therefore comprises a base and side walls to hold a container 118 placed therein and thereatop. The basin sidewalls may be conformably sized and shaped according to the size and shape of the container 118.

In the embodiment shown, each container 118 comprises a container body 124 and a releasable lid 125 engaged thereto. Furthermore, in the embodiment shown, the container 118 is orientated vertically. The configuration of the shelf 121 allows each container 118 to be easily held by opposite lateral sides above the partitions 122 in one hand and retrieved from the cradle 115.

The rear of the cradle 115 may comprise a sensor recess 123 engaging a sensor 117. With reference to FIGS. 3 and 4, each shelf 121 may be supported from a wall mount panel 116.

With reference to FIG. 3, the sensor 118 may comprise a light beam interrupt sensor having a transmitter 127 which transmits a beam of light 129 received by a receiver 130. As such, placement of the container 118 within the cradle 115 interrupts the beam of light 129, thereby allowing the controller to detect the presence of the container 118 therein.

With reference to FIG. 1, the computer program code controllers may comprise a sensor controller 108 configured for interfacing with the various sensors 117 for detecting the presence of the containers 118 therein. As such, in accordance with the embodiment of FIG. 3, the sensor controller 108 may be configured such that the loss of signal by the receiver 130 is indicative of the placement of the container 118 within the respective cradle 115.

FIG. 4 shows an alternative embodiment wherein the sensor 118 comprises an electromechanical contact switch 131. In accordance with this embodiment, the switch 131 comprises a switch lever 132 which protrudes through the sensor recess 123. The lever 132 electromechanically engages a switch body 133 so as to open or close an electrical circuit formed by electrical leads 134 connected thereto. When the container 118 is placed within the cradle 115, the lever 132 is depressed and, alternatively, when the container 118 is removed from the cradle 115, the lever 132 rises, thereby allowing the sensor controller 100 to detect the presence of the container 118 by the open or closed state of the switch 132.

FIG. 5 shows an embodiment wherein the sensor comprises a radiofrequency receiver 135 configured for detecting the presence of radiofrequency tags 136 of each container 118 within proximity.

In embodiments, the radiofrequency receiver 135 is configured to detect the presence of the radiofrequency tags 136 within proximity of approximately 1 m for example. As such, the removal of the container 118 beyond this range allows the sensor controller 100 and to detect the absence of the particular container.

In embodiment, the radiofrequency receiver 135 reads data encoded within the radiofrequency tags 136, including, in embodiments, container type and container identity. Additional information may be encoded within the radiofrequency tags 136 relating to the contents within the container 118, including the first-aid kit items therein, associated expiry dates, contraindications and the like.

In embodiments, the system 100 comprises a plurality of different types of containers, each of which are uniquely physically keyed with a corresponding cradle 115 such that each type of container can only be inserted within a corresponding cradle. As such, the containers 118 within the rack 116 may be configured for storing a range of essential first-aid kit types and, furthermore, the controller 106 may be configured for monitoring the presence or absence of each type of container using a respective sensor 117 within each respective cradle 115.

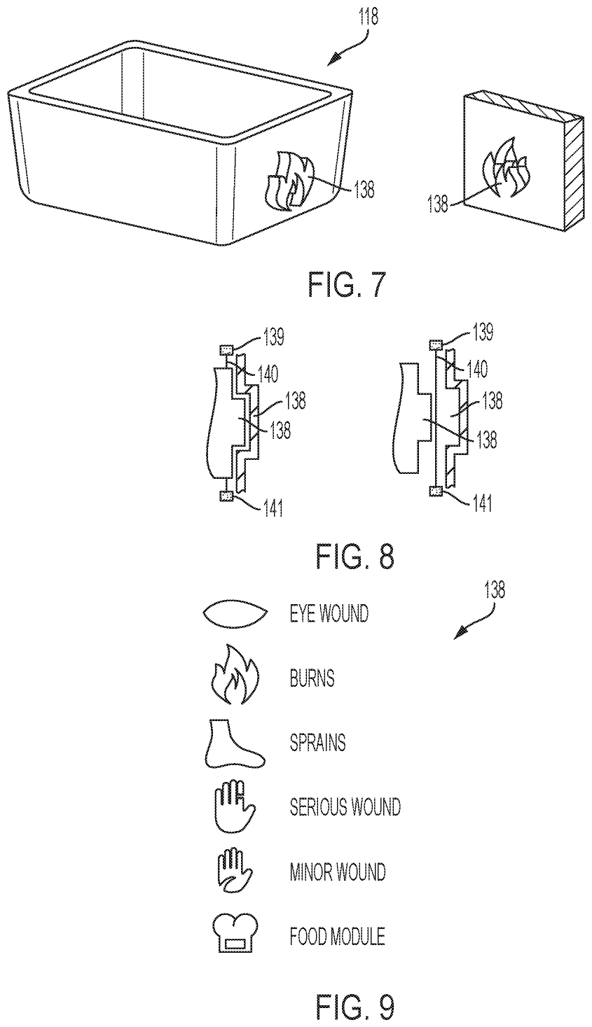

In embodiments of FIG. 7, the container 118 comprises a keying formation 138 on a surface thereof and the corresponding cradle surface comprises a corresponding keying formation 138 which align and uniquely mate together when the container 118 is placed within the correct cradle 115. In the embodiment shown, the formations 138 of the container 118 is a male formation and the formation 137 of the cradle 115 is a female formation.

In embodiments, the formations 138 may comprise different shapes as illustrated in FIG. 9 to visually illustrate the use of the purposes of each container 118.

FIG. 8 shows an embodiment wherein a light beam interrupt is used to detect the insertion of the male formation 138 within the female formation 138. Specifically, the interrupt may comprise a light transmitter 139 configured for directing a beam of light 140 towards a receiver 141. The beam of light is directed closely across the surface of the cradle 115 such that when the male formation 138 inserts within the female formation 138, the light beam 140 is interrupted, thereby allowing the sensor controller 108 to detect the presence of the container 118.

In embodiments, the transmitter and the receiver 139, 141, are located behind the rear panel 126 and which direct the beam of light through the female formation 138 by appropriately positioned entrance and exit slots.

Exemplary utilisation of the system 100 will now be described with reference to an illustrative embodiment.

A rack 116 may be placed within a suitable location, such as a workplace environment or the like and which may be wall mounted using the wall mount panel 126. A plurality of containers 118 may be filled with first aid items for various purposes, such as for my wounds, burns, sprains, serious wounds, minor wounds and the like.

In embodiments, the controller 106 stores inventory data 109 used by an associated inventory controller to the inventory of each container 118.

In embodiments, each container 118 is sealed container 118 and the contents thereof stored in the inventory data 109.

In embodiments, the controller 106 may transmit an identity or type of a container 118 to a server 103 so as to receive data indicative of the contents therein and other associated data including data indicative of the purpose of the container, instructional data (as will be described in further detail below) and the like. Retrieval of such data from the server 103 allows the controller 106 to use different types of containers 118, including those whose contents change over time.

Each container 118 is loaded within the respective cradles 115, thereby being ready for use.

In embodiments, the controller monitors the shelf life of contents within each container 118 in accordance with the inventory data 109 and provides periodic notifications indicative of the requirement for the replenishment or replacement thereof.

For example, the indicator panel 119 may indicate which container 118 requires replacement. The indicator panel 119 may comprise a plurality of LEDs, each of which can be cross-referenced with a particular cradle 115. Alternatively, a LED may be placed adjacent each cradle 115. A green light may indicate that the container 118 is ready for use whereas an orange light may indicate that the container 118 is about to expire and the red light may indicate that the container 118 has expired.

Each container 118 may have a different shelf life depending on the contents therein. Generally, the shelf life of each container 118 will be set in accordance with the shortest shelf life of any one of the items contained therein. As such, the containers 118 may be replaced periodically such that all of the contents therein remain within their respective shelf lives.

In embodiments, each container 118 may be encoded with a container type and a unique container identity. As such, the controller 106 is able to determine when a particular container 118 is replaced by detecting a change in the container identity. Alternatively, each container 118 may encode a packaging date and or and expiry date. As such, the controller 106 is able to read the relevant date from the container 118 and thereafter monitor the expiry thereof in relation to the date.

For example, for a hand burn, as such, a burns-type container 118 may be retrieved from the rack 116 for treatment.

In embodiments, each container 118 may be labelled with the purpose of each container allowing the user to quickly identify the appropriate container.

A user may use the user interface 121 to select or input the type of injury against which the controller 106 performs a search against the inventory data 109 to update the interface 121 with an indication of one or more appropriate matching containers 118 for use.

In embodiments, the audio input device 130 may receive voice data and perform speech recognition thereon so as to identify the appropriate container 118. For example, the user may stand in front of the rack 116 and announce “I have burnt my hand”. The audio input device 113 may encode the voice of the user and the controller 106 may transmit the audio data to the server 103 to perform speech recognition thereon. The server 103 returns the text of the speech recognised, allowing the controller 106 to perform a search against the inventory data 109 to indicate the appropriate container 118, such as by illuminating a respective LED thereof.

Once having identified the appropriate container 118, the user retrieves the appropriate container 118 from the rack 116. Where the container 118 comprises a lid 125, the user may remove the lid 125 from the container body 124 to access the contents therein.

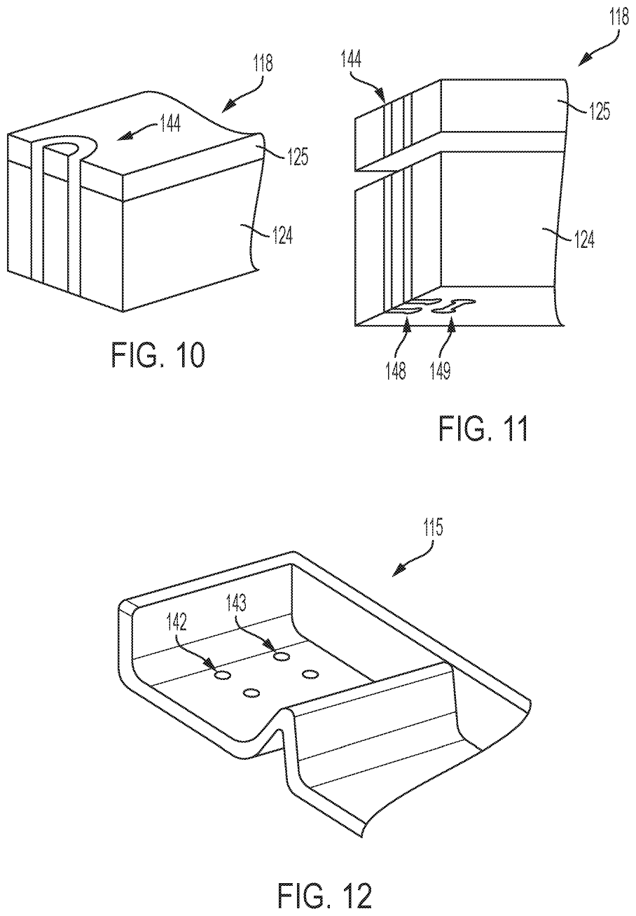

In the embodiment shown in FIGS. 10 and 11 a circuit completing foil strip 144 interfaces the lid 125 and the container body 124. As such, removing the lid 125 from the container body 124 tears the foil strip 144. The foil strip may expose electrical contacts 148 which locate over corresponding electrical contacts 143 of the cradle 115 when the container 118 locates within the cradle 118.

As such, whereas electric current may normally flow through the circuit completing foil strip when intact, the tearing thereof by the removal of the lid 125 breaks the electrical circuit thereby allowing the controller 106 to ascertain that the lid 125 has been removed from the container body 124 therefore that the items therein require replenishment. The system 100 may allow the removal and replacement of containers 118 without opening the containers, and thereby without requiring replacement of the contents therein.

As is shown in FIG. 11, the container 115 may further comprise an additional foil strip 149 which completes a circuit with corresponding electrical contacts 142 within the cradle 115.

As such, the controller 106 may use the electrical contacts 142 to detect the presence of the container 118 within the cradle and the further electrical contacts 143 to detect the opening of the lid 125.

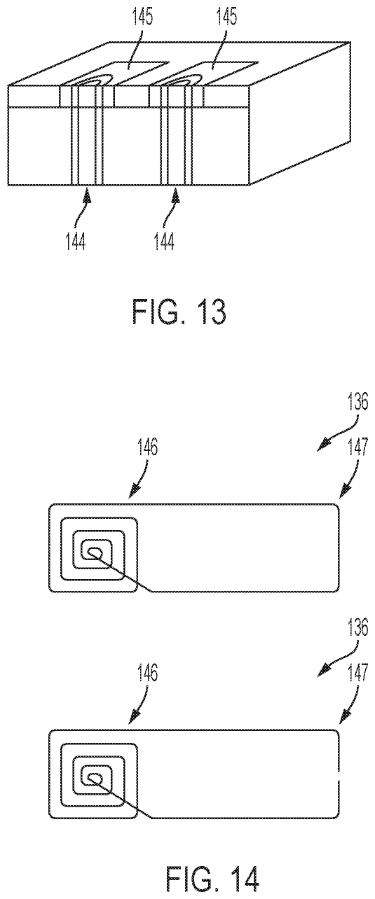

In embodiments, the container 118 comprises a separate electric circuit for each first-aid item therein. In the example shown in FIG. 13 the container 118 may comprise a plurality of compartments 145 each having a lid across which a foil strip 144 is run and which is broken by the opening of each compartment lid. As such, depending on which separate electric circuit is broken, the controller 118 is able to ascertain which first-aid item has been used or removed from the container.

Each cradle 115 may comprise electrical contacts for each separate electric circuit of each container 118.

Alternatively, for noncontact sensing, as is shown a FIG. 14 each container 118 comprises a plurality of radiofrequency tags, antennas 146 thereof connected in series with a plurality of electric circuits 147 and wherein each electric circuit breaks when a respective first-aid item is accessed thereby disabling a respective radiofrequency tag 146. As such the controller 106 is configured for using a radio frequency receiver to detect which of the plurality of radio frequency tags are disabled to identify which first-aid items have been accessed.

Each cradle 118 may comprises an indicator and the controller 106 may be configured for controlling the indicator to indicate that the at least one first-aid item requires replenishment when detecting the removal of the container lid or access of a first-aid item.

In embodiments, the system 100 is configured for outputting instructional information guiding the user as to the appropriate use of the first-aid items within the container 118, thereby allowing use of the first-aid items with little or no medical knowledge.

In embodiments, the inventory data 109 may comprise a contraindication data such that user interface 121 is able to indicate medicament contraindications, including user specific contraindications or contraindications with other medicaments.

In embodiments, the user interface 121 may control a decision tree user interface allowing the user to step through to the appropriate instructions. For example, the user interface 121 may allow the user to input the location of the burn and thereafter the severity thereof. In accordance with such user input, the interface 121 may then display information as to how to apply a bandage to a hand to address a minor burn.

In additional or alternative embodiments, the audio output device 114 may output audio instructional information indicative of the appropriate use of the first-aid items.

In embodiments, a user mobile device 101 may be paired or connected with the controller 106 so as to be able to interact with the controller 106 for the provision of instructional information and receipt of user feedback accordingly.

In embodiments, the controller 106 comprises a notification controller 111 configured for sending a notification data across the wide area network 104 such as to one or more be configured mobile devices 101 or servers 103.

For example, in one embodiment, the removal of the “burns” first aid container 118 from the rack 116 is identified by the sensor controller 108 which causes the notification controller 111 to send a notification to one or more pre-configured mobile contact numbers of respective mobile devices 101. In embodiments, the contact numbers may be configured using user interface device 121. In further embodiments, the notification controller 111 may be configured only for sending a notification in accordance with user feedback received by the user interface 121 or audio input 113 so as to, for example, only send notifications for severe burns as opposed to minor burns.

In embodiments, the notification controller 111 may transmit an electronic communication to an electronic communication device upon removal of the container 118 which comprises instructional information instructing the user as to the appropriate utilisation of the first-aid kit contents therein which may be displayed on the electronic display device of the mobile phone device. In embodiments, the electronic communication may be a push notification transmitted to a mobile phone of a particular user registered with the system 100 or a mobile phone determined as being within proximity of the system 100, such as, for example, the proximity thereof being detected by the radiofrequency receiver 135.

In alternative embodiments, the notification controller 111 may transmit an electronic communication such as an email or push notification to an electronic communication device associated with a particular container 118.

In embodiments, the system 100 may comprise a backup power supply and/or lighting such that, in the event of loss of power or lighting, the system 100 may continue operation and, in embodiments, provide illumination in the dark. During loss of power, the notification controller 111 may transmit an electronic communication to a predetermined destination.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

1. A first-aid kit dispensing system comprising a plurality of cradles for a respective plurality of first-aid kit containers, each container having a container body and container lid a plurality of first-aid kit items therein, at least one sensor configured for detecting the presence of the containers, a controller operably coupled to the at least one sensor, the controller comprising a memory device recording an inventory of a plurality of first aid items and wherein, in use, the controller is configured for using the at least one sensor to detect at least one of a container identity and a container type of a container inserted into or removed from a container and accessing the inventory data to identify at least one first aid item of the container.

2. The first-aid kit dispensing system as claimed in claim 2, wherein each container comprises a container lid and a container body and wherein the system is configured for detecting the removal of the container lid from the container body.

3. The first-aid kit dispensing system as claimed in claim 2, wherein the controller is configured for updating the inventory data to indicate that the at least one first-aid item requires replenishment when detecting the removal of the container lid.

4. The first-aid kit dispensing system as claimed in claim 2, wherein each cradle comprises an indicator and wherein the controller is configured for controlling the indicator to indicate that the at least one first-aid item requires replenishment when detecting the removal of the container lid.

5. The first-aid kit dispensing system as claimed in claim 2, wherein each container comprises an electrical circuit interfacing the container lid and the container body and wherein opening of the container lid breaks the electrical circuit and wherein the system is configured for detecting breaking of the electrical circuit.

6. The first-aid kit dispensing system as claimed in claim 5, wherein the electrical circuit comprises tearable foil strips interfacing the container lid and the container body which are torn when the lid is removed from the body.

7. The first-aid kit dispensing system as claimed in claim 5, wherein the cradle comprises electrical contacts which make electrical contact with the electrical circuit when a respective container is placed therein.

8. The first-aid kit dispensing system as claimed in claim 1, wherein the container comprises an electric circuit for each first-aid item therein and wherein each electric circuit breaks when a respective first-aid item is accessed and wherein the controller is configured for determining which first-aid items have been accessed accordingly.

9. The first-aid kit dispensing system as claimed in claim 8, wherein the cradle comprises electrical contacts for each electric circuit.

10. The first-aid kit dispensing system as claimed in claim 1, wherein corresponding containers and cradles are keyed such that each container can only be inserted into a correspondingly keyed cradle and wherein the controller is configured for identifying the at least one of the container identity and container type depending on which container as inserted into or removed from a particular container.

11. The first-aid kit dispensing system as claimed in claim 10, wherein each keyed container and correspondingly keyed cradle comprises male and female keying formations on respective abutting surfaces thereof which interlock when the keyed container is inserted into the correspondingly keyed cradle.

12. The first-aid kit dispensing system as claimed in claim 11, wherein the at least one sensor comprises a plurality of sensors for each cradle and wherein each of the plurality of sensors is configured for detecting interlock of the keying formations.

13. The first-aid kit dispensing system as claimed in claim 12, wherein each sensor comprises a light beam interruption sensor operative across the respective key information.

14. The first-aid kit dispensing system as claimed in claim 1, wherein each cradle comprises a base and edges sized and shaped according to a size and shape of the container.

15. The first-aid kit dispensing system as claimed in claim 1, wherein the at least one sensor comprises a plurality of sensors for each cradle and wherein each sensor comprises a light beam interrupt sensor configured for detecting interruption of a beam of light by a container inserted within a respective cradle.

16. The first-aid kit dispensing system as claimed in claim 1, wherein the at least one sensor comprises a plurality of sensors for each cradle and wherein each sensor comprises an electromechanical switch comprising a switch lever which extends into the cradle and which is deflected by the container when located within the cradle for being indicative of the presence of the container.

17. The first-aid kit dispensing system as claimed in claim 1, wherein each container comprises computer readable media encoding the at least one of the container identity and container type and wherein the controller is configured for interrogating the computer readable media using a radiofrequency receiver to read the at least one of the container identity and container type therefrom.

18. The first-aid kit dispensing system as claimed in claim 17, wherein each container comprises a radiofrequency tag and the sensor comprises a radiofrequency receiver configured for reading the radiofrequency tags.

19. The first-aid kit dispensing system as claimed in claim 1, wherein the container comprises a plurality of radiofrequency tags, antennas thereof connected in series with a plurality of electric circuits and wherein each electric circuit breaks when a respective first-aid item is accessed thereby disabling a respective radiofrequency tag and wherein the controller is configured for using a radio frequency receiver to detect which of the plurality of radio frequency tags are disabled to identify which first-aid items have been accessed.

20. The first-aid kit dispensing system as claimed in claim 1, wherein the controller is configured for monitoring an expiry date of each container.

21. The first-aid kit dispensing system as claimed in claim 20, wherein the controller is configured for calculating the expiry date in accordance with the container identity or container type.

22. The first-aid kit dispensing system as claimed in claim 20, wherein the controller is configured for reading an expiry date encoded within computer readable medium of the container.

23. The first-aid kit dispensing system as claimed in claim 20, wherein the controller is configured for using the at least one sensor for recording and insertion date when the container is inserted within a respective cradle and a calculating the expiry date accordingly.

24. The first-aid kit dispensing system as claimed in claim 20, wherein each cradle comprises an indicator and wherein the controller is configured for controlling the indicator according to the expiry date of each respective container.

25. The first-aid kit dispensing system as claimed in claim 24, wherein the indicator panel comprises an LED adjacent each cradle and wherein each LED is controllable in at least two states indicative of at least two shelf life states of each container.

26. The first-aid kit dispensing system as claimed in claim 1, wherein the system comprises a user interface and wherein, in use, the controller is configured for receiving data via the user interface and identifying at least one container accordingly.

27. The first-aid kit dispensing system as claimed in claim 26, wherein the user interface comprises a drill down interface allowing for the further input of specifics of a first-aid situation and wherein the controller is configured identifying at least one container according to the specifics.

28. The first-aid kit dispensing system as claimed in claim 26, wherein the system further comprises a user interface and wherein, in use, the controller is configured for detecting a removal of a container and outputting instructional information for the use of the at least one container.

29. The first-aid kit dispensing system as claimed in claim 1, wherein the system further comprises a notification controller and, wherein, in use, the controller is configured for identifying at transmitting an electronic notification according to the at least one of the container identity and the container type of the container.
